(19) United States
(12) Patent Application Publication
(10) Pub. No.: US 2017/0042644 A1
(43) Pub. Date: Feb. 16, 2017

(54) IMPLANT AND MANUFACTURING METHOD THEREFOR

(71) Applicant: OLYMPUS CORPORATION, Tokyo (JP)

(72) Inventor: Masato TAMAI, Tokyo (JP)

(73) Assignee: OLYMPUS CORPORATION, Tokyo (JP)

(21) Appl. No.: 15/335,559

(22) Filed: Oct. 27, 2016

Related U.S. Application Data
(63) Continuation of application No. PCT/JP2015/056113, filed on Mar. 2, 2015.

(30) Foreign Application Priority Data
Jun. 5, 2014 (JP) 2014-116953

(51) Int. Cl. A61C 8/00; A61C 8/02; C25D 11/30; A61L 27/04; A61L 27/12; A61L 27/56; A61F 2/28; A61C 13/00

(52) U.S. Cl. CPC A61C 8/0015; A61F 2/28; A61C 8/0006; A61C 13/0012; A61L 27/047; A61L 27/12; A61L 27/56; C25D 11/30; A61F 2310/00041; A61L 2430/12

(57) ABSTRACT

Provided is an implant including a base material composed of magnesium or a magnesium alloy and an anodized membrane formed on a surface of the base material. The anodized membrane has 8,000 to 250,000 pores with an average diameter of 0.1 μm to 1 μm within 1 mm².

### CROSS-REFERENCE TO RELATED APPLICATIONS

This is a continuation of International Application PCT/JP2015/056113 which is hereby incorporated by reference herein in its entirety.

This application is based on Japanese Patent Application No. 2014-116953, the contents of which are incorporated herein by reference.

### TECHNICAL FIELD

The present invention relates to implants and manufacturing methods therefor.

### BACKGROUND ART

A popular implant treatment in the related art involves implanting an implant composed of titanium or a titanium alloy into the jawbone with a missing tooth and directly joining the implant to the bone (osteo-integration) so that the implant substitutes for a natural root of a tooth. In order to give a dental implant a surface that is easily joinable to bone tissue, it is known to reform the surface by blasting, acidizing, or anodizing (for example, see Patent Literatures 1 and 2).

According to Patent Literatures 1 and 2, pores on the order of several tens of micrometers in the surface of the implant have an effect of increasing the surface area thereof to increase the contact area with the bone tissue. Pores that are 1 μm to 2 μm are known to have an effect of maintaining blood-derived fibrin fibers in the implant surface, and pores that are several tens to several hundreds of nanometers are known to have an effect of increasing the adhesive force of cells, bone-active substances from osteoblastic cells, and the deposition amount of calcium.

For treatment of fractures, biodegradable osteosynthetic materials that are composed of magnesium alloys and are biodegraded within the body have been developed. As an ideal function of a biodegradable osteosynthetic material, it is demanded that the material replace bone while decomposing. In a known biodegradable osteosynthetic material of this type, an anodized membrane is formed on the surface thereof to suppress tissue damage caused by hydrogen gas generated when the magnesium alloy is decomposed (for example, see Patent Literature 3).

### CITATION LIST

### Patent Literature

### {PTL 1}

### Japanese Translation of PCT International Application, Publication No. 2003-500160

### {PTL 2}

### Japanese Unexamined Patent Application, Publication No. 2012-143416

### {PTL 3}

PCT International Publication No. WO 2013/070669

### SUMMARY OF INVENTION

### Solution to Problem

An aspect of the present invention provides an implant including a base material composed of magnesium or a magnesium alloy and an anodized membrane formed on a surface of the base material. The anodized membrane has 8,000 to 250,000 pores with an average diameter of 0.1 μm to 1 μm within 1 mm².

Another aspect of the present invention provides a method for manufacturing an implant. The method includes performing an anodizing process involving immersing a base material composed of magnesium or a magnesium alloy in an electrolytic solution with a pH value ranging between 9 and 13 and containing 0.1 mol/L or smaller of phosphoric acid and 0.2 mol/L of ammonia or ammonium ions but not containing elemental fluorine and applying electricity to the base material, so as to form an anodized membrane having 8,000 to 250,000 pores of 0.1 μm to 1 μm within 1 mm² on a surface of the base material.

### BRIEF DESCRIPTION OF DRAWINGS

FIG. 1 is a partial vertical sectional view illustrating an implant according to an embodiment of the present invention.

FIG. 2 illustrates an electron-microscope image showing a first example of the implant in FIG. 1.

FIG. 3 illustrates an electron-microscope image showing a second example of the implant in FIG. 1.

FIG. 4 includes a microscope image (a) showing a state where the implant in FIG. 3 is implanted within a biological organism and an enlarged image (b) of (a).

FIG. 5 illustrates an electron-microscope image showing a third example of the implant in FIG. 1.

### DESCRIPTION OF EMBODIMENTS

An implant and a manufacturing method therefor according to an embodiment of the present invention will be described below with reference to the drawings.

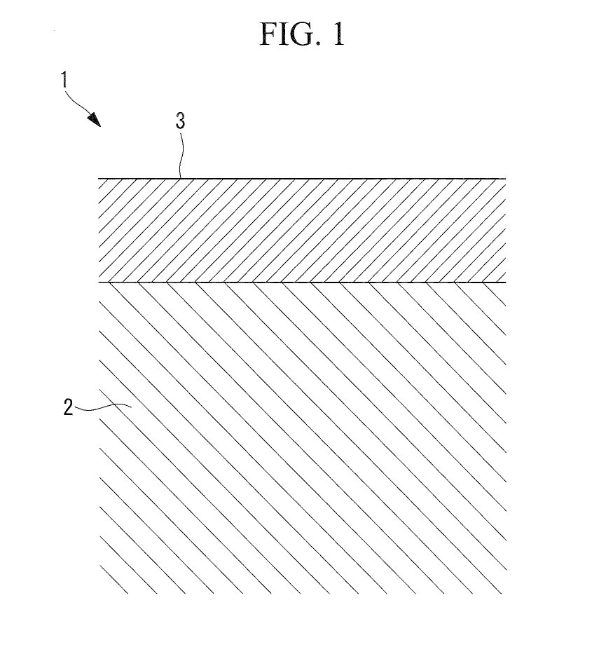

As shown in FIG. 1, an implant 1 according to this embodiment includes an anodized membrane 3 on the surface of a base material 2 composed of magnesium or a magnesium alloy.

The base material 2 may be composed of any material having magnesium as a main component and may be composed of metal containing magnesium alone or may be composed of a magnesium alloy. In order to give the base material 2, for example, moldability, mechanical strength, and ductility, a magnesium alloy is used. Examples of the magnesium alloy include a Mg—Al alloy, a Mg—Al—Zn alloy, a Mg—Al—Mn alloy, a Mg—Zn—Zr alloy, a Mg-rare-earth-element alloy, and a Mg—Zn-rare-earth-element alloy.

The anodized membrane 3 has 8,000 to 250,000 pores with an average diameter of 0.1 μm to 1 μm within 1 mm².

A method for manufacturing the implant 1 according to this embodiment having the above-described configuration is as follows.

Specifically, the implant 1 according to this embodiment is manufactured by performing an anodizing process involving immersing the base material in an electrolytic solution with a pH value ranging between 9 and 13 and containing 0.1 mol/L or smaller of phosphoric acid and 0.2 mol/L of ammonia or ammonium ions but not containing elemental fluorine, and applying electricity to the base material.

The anodizing process is performed by connecting a power source between the base material 2 immersed in the electrolytic solution and serving as an anode and a cathode material similarly immersed in the electrolytic solution.

Although the power source used is not limited in particular and may be a direct-current power source or an alternating-current power source, a direct-current power source is preferred.

In the case where a direct-current power source is used, it is preferable that a constant-current power source be used. The cathode material used is not limited in particular. For example, stainless steel may be suitably used. The surface area of the cathode is preferably larger than the surface area of the magnesium alloy to be anodized.

The electric current density at the surface of the base material 2 serving as an anode when a constant-current power source is used as the power source is 20 A/dm² or higher. The electricity application time is between 10 seconds and 1000 seconds. When electricity is to be applied by using a constant-current power source, the applied voltage is low at the start of the electricity application process but increases as time elapses. The final applied voltage when the electricity application process ends is 350 V or higher.

In the implant 1 manufactured in this manner, the anodized membrane 3 at the surface thereof has 8,000 to 250,000 pores with an average diameter of 0.1 μm to 1 μm within 1 mm².

Pores of 1 μm in size have an effect of maintaining fibrin fibers at the surface of the implant 1, and pores on the order of 0.1 μm have an effect of increasing the adhesive force of cells, bone-active substances from osteoblastic cells, and the deposition amount of calcium. Therefore, the implant 1 according to this embodiment can enhance the osteo-integration performance.

Then, after direct fusion between the bone tissue and the implant 1 caused by osteo-integration, the base material 2 is biodegraded. Consequently, the implant 1 does not remain as a foreign object within a biological organism over a long period of time, thus eliminating the need for performing a removal process.

### Examples

### First Example

Next, a first example of an implant 1 according to an embodiment of the present invention will be described.

In the implant 1 according to this example, the anodized membrane 3 formed on the surface of the base material 2 composed of a magnesium alloy has 56,000 pores with an average diameter of 1 μm within 1 mm².

The base material 2 is immersed in an electrolytic solution with 0.05 mol/L of phosphoric acid. By using a constant-current power source with an electric current density of 20 A/dm² at the anode surface as a power source, electricity is applied for 60 seconds. The final applied voltage when the electricity application process ends is 400 V.

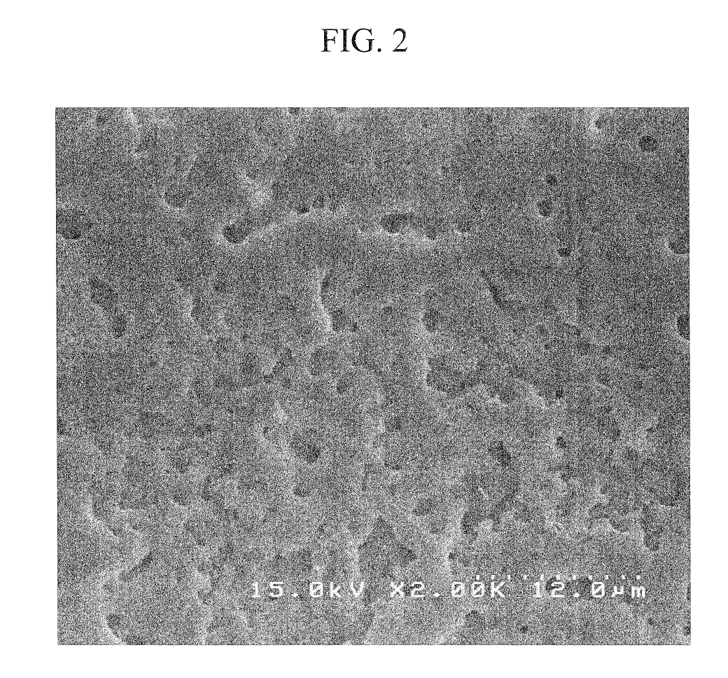

An electron-microscope image of the anodized membrane 3 at the surface of the implant 1 manufactured in this manner is shown in FIG. 2. According to this, every 1 mm² region has 56,000 pores with a diameter of 0.4 μm to 5 μm and an average diameter of 1 μm.

According to this example, the anodized membrane 3 at the surface of the implant 1 can maintain the fibrin fibers at the surface of the implant 1 by means of the pores with the average diameter of 1 μm.

The components of the anodized membrane 3 are as shown in Table 1 below.

TABLE 1

| Element | Mass Concentration(%) |
|---|---|
| CK | 3.58 |
| OK | 48.08 |
| MgK | 23.83 |
| PK | 21.58 |
| PtM | 2.93 |
| Total | 100.00 |

### Second Example

Next, a second example of an implant 1 according to an embodiment of the present invention will be described.

In the implant 1 according to this example, the anodized membrane 3 formed on the surface of the base material 2 composed of a magnesium alloy has 62,000 pores with an average diameter of 0.5 μm within 1 mm².

The base material 2 is immersed in an electrolytic solution with 0.1 mol/L of phosphoric acid. By using a constant-current power source with an electric current density of 30 A/dm² at the anode surface as a power source, electricity is applied for 60 seconds. The final applied voltage when the electricity application process ends is 350 V.

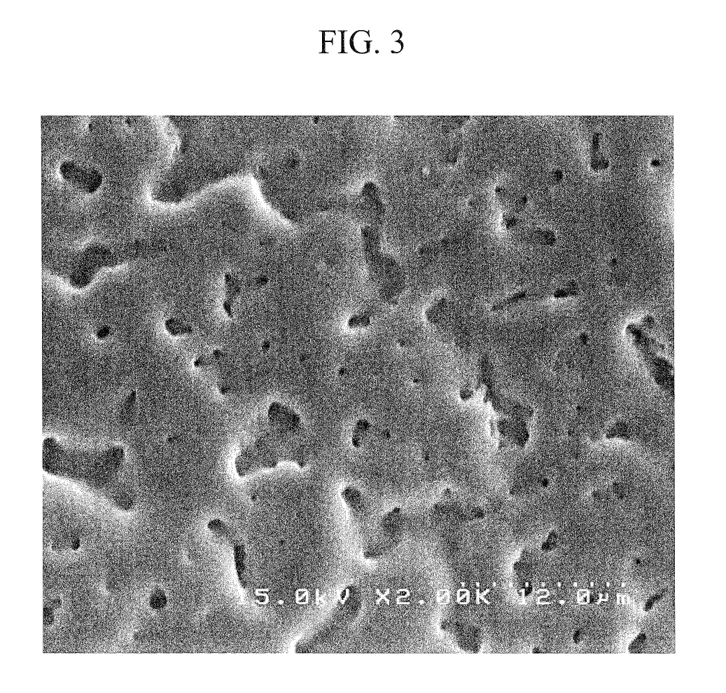

An electron-microscope image of the anodized membrane 3 at the surface of the implant 1 manufactured in this manner is shown in FIG. 3. According to this, every 1 mm² region has 62,000 pores with a diameter of 0.2 μm to 1.2 μm and an average diameter of 0.5 μm. Furthermore, pores (protrusions and recesses) that are about 10 μm, which are considered to be processing marks, are formed over the entire surface of the implant 1.

According to this example, the anodized membrane 3 at the surface of the implant 1 can maintain the fibrin fibers at the surface of the implant 1 by means of the pores with the average diameter of 0.5 μm.

The components of the anodized membrane 3 are as shown in Table 2 below.

TABLE 2

| Element | Mass Concentration (%) |
|---|---|
| CK | 3.73 |
| OK | 46.97 |
| MgK | 23.84 |
| PK | 21.51 |
| PtM | 3.95 |
| Total | 100.00 |

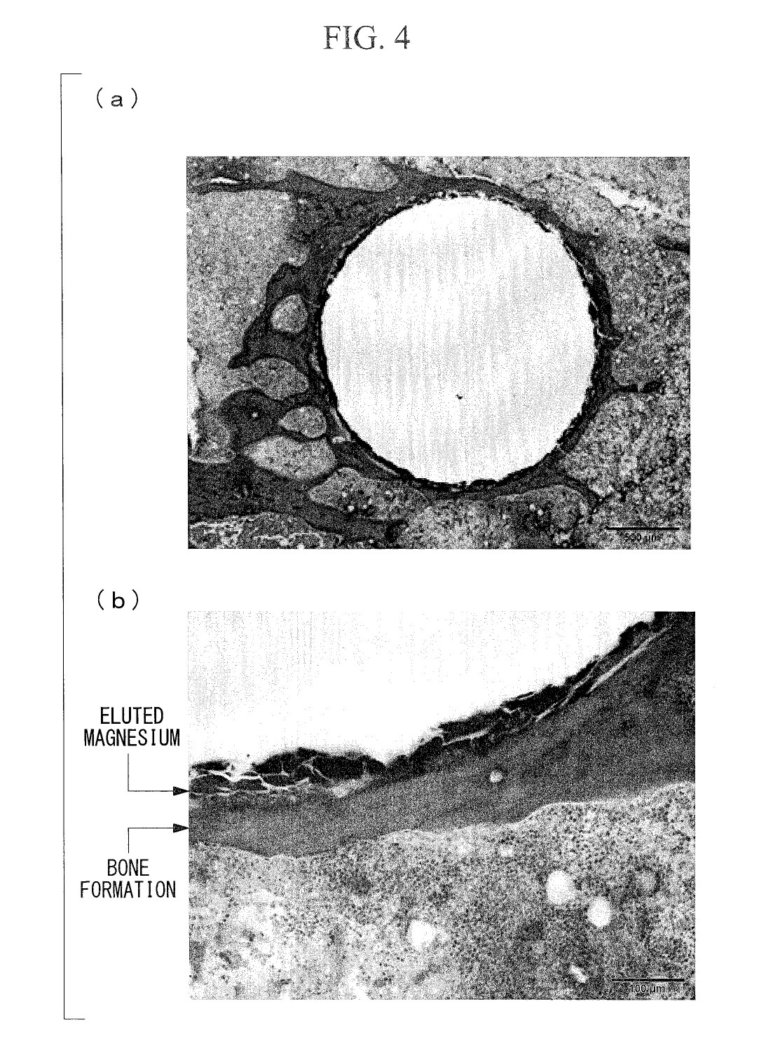

The implant 1 manufactured in this manner implanted in a bone of a rat, and a microscope image obtained after three months is shown in FIG. 4. The white circle at the center of FIG. 4(a) corresponds to the implant 1 according to this example. According to FIG. 4(a) and FIG. 4(b), which is an enlarged view thereof, it can be confirmed that the cells adhere to each other so that a magnesium oxide or magnesium phosphate elutes around the implant 1 and that bone formation starts therearound.

### Third Example

Next, a third example of an implant 1 according to an embodiment of the present invention will be described.

In the implant 1 according to this example, the anodized membrane 3 formed on the surface of the base material 2 composed of a magnesium alloy has 248,520 pores with an average diameter of 100 nm within 1 mm².

The base material 2 is immersed in an electrolytic solution with 0.05 mol/L of phosphoric acid. By using a constant-current power source with an electric current density of 30 A/dm² at the anode surface as a power source, electricity is applied for 60 seconds. The final applied voltage when the electricity application process ends is 350 V.

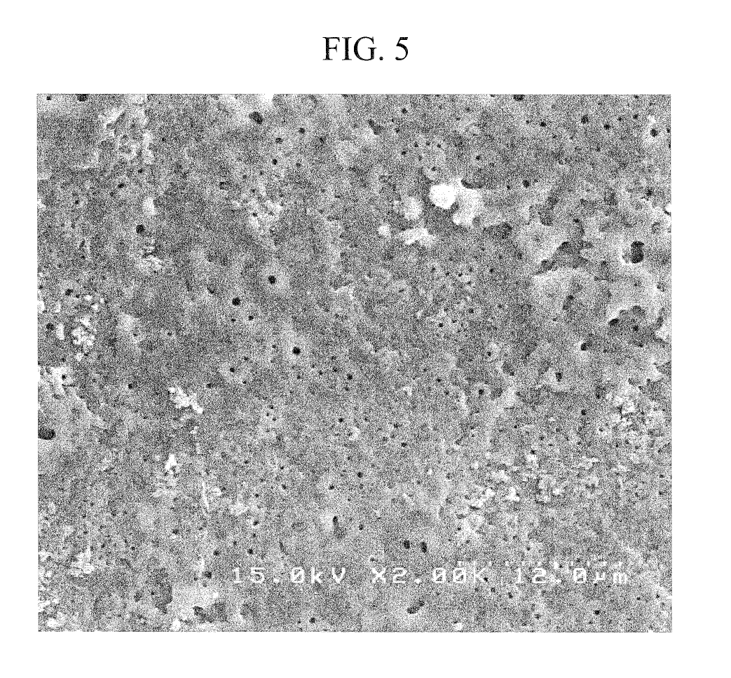

An electron-microscope image of the anodized membrane 3 at the surface of the implant 1 manufactured in this manner is shown in FIG. 5. According to this, every 1 mm² region has 248,520 pores with a diameter of 50 nm to 200 nm and an average diameter of 100 nm.

According to this example, the anodized membrane 3 at the surface of the implant 1 can maintain the fibrin fibers at the surface of the implant 1 by means of the pores with the average diameter of 100 nm, and the adhesive force of cells, bone-active substances from osteoblastic cells, and the deposition amount of calcium can be increased.

The components of the anodized membrane 3 are as shown in Table 3 below.

TABLE 3

| Element | Mass Concentration (%) |
|---|---|
| OK | 45.74 |
| MgK | 24.36 |
| PK | 22.16 |
| Others | 3.56 |
| Total | 100.00 |

The above-described embodiment leads to the following inventions.

An aspect of the present invention provides an implant including a base material composed of magnesium or a magnesium alloy and an anodized membrane formed on a surface of the base material. The anodized membrane has 8,000 to 250,000 pores with an average diameter of 0.1 μm to 1 μm within 1 mm².

According to this aspect, pores of 1 μm in size formed in the anodized membrane have an effect of maintaining fibrin fibers at the surface, and pores on the order of 0.1 μm can increase the adhesive force of cells, bone-active substances from osteoblastic cells, and the deposition amount of calcium, thereby enhancing the osteo-integration performance.

In the above aspect, the anodized membrane may have 8,000 to 62,000 pores with an average diameter of 0.5 μm to 1 μm within 1 mm².

Accordingly, since there are a small number of pores on the order of 0.1 μm, the adhesive force of cells is weakened, thereby allowing for improved removability when a problem occurs.

Furthermore, in the above aspect, the anodized membrane may have 62,000 to 250,000 pores with an average diameter of 0.1 μm to 0.5 μm within 1 mm².

Accordingly, the adhesive force of cells, bone-active substances from osteoblastic cells, and the deposition amount of calcium can be increased, so that the osteo-integration performance can be sufficiently enhanced even for a patient with poor bone formation.

Furthermore, in the above aspect, the anodized membrane may have pores each having a diameter of 10 μm or larger.

Accordingly, with the pores that are 10 μm or larger, the contact area with the bone tissue is increased, so that a large number of osteoblastic cells are accumulated, thereby increasing the deposition amount of calcium.

Furthermore, in the above aspect, the anodized membrane may contain 20% to 30% by weight of magnesium element, 40% to 50% by weight of oxygen element, and 10% to 30% by weight of phosphorus element and may be formed by performing an anodizing process in an electrolytic solution having a phosphoric acid concentration of 0.1 mol/L or smaller.

Accordingly, the anodized membrane is biodegraded within the body, the fibrin fibers are maintained, and the adhesive force of cells, bone-active substances from osteoblastic cells, and the deposition amount of calcium can be increased.

Another aspect of the present invention provides a method for manufacturing an implant. The method includes performing an anodizing process involving immersing a base material composed of magnesium or a magnesium alloy in an electrolytic solution with a pH value ranging between 9 and 13 and containing 0.1 mol/L or smaller of phosphoric acid and 0.2 mol/L of ammonia or ammonium ions but not containing elemental fluorine and applying electricity to the base material, so as to form an anodized membrane having 8,000 to 250,000 pores of 0.1 μm to 1 μm within 1 mm² on a surface of the base material.

### REFERENCE SIGNS LIST

1 implant
2 base material
3 anodized membrane

1. An implant comprising:
a base material composed of magnesium or a magnesium alloy; and
an anodized membrane formed on a surface of the base material,
wherein the anodized membrane has 8,000 to 250,000 pores with an average diameter of 0.1 μm to 1 μm within 1 mm².

2. The implant according to claim 1,
wherein the anodized membrane has 8,000 to 62,000 pores with an average diameter of 0.5 μm to 1 μm within 1 mm².

3. The implant according to claim 1,
wherein the anodized membrane has 62,000 to 250,000 pores with an average diameter of 0.1 μm to 0.5 μm within 1 mm².

4. The implant according to claim 1,
wherein the anodized membrane has pores each having a diameter of 10 μm or larger.

5. The implant according to claim 1,
wherein the anodized membrane contains 20% to 30% by weight of magnesium element, 40% to 50% by weight of oxygen element, and 10% to 30% by weight of phosphorus element and is formed by performing an anodizing process in an electrolytic solution having a phosphoric acid concentration of 0.1 mol/L or smaller.

6. A method for manufacturing an implant, comprising:
performing an anodizing process involving immersing a base material composed of magnesium or a magnesium alloy in an electrolytic solution with a pH value ranging between 9 and 13 and containing 0.1 mol/L or smaller of phosphoric acid and 0.2 mol/L of ammonia or ammonium ions but not containing elemental fluorine and applying electricity to the base material, so as to form an anodized membrane having 8,000 to 250,000 pores of 0.1 μm to 1 μm within 1 mm² on a surface of the base material.
